(19) United States
(12) Patent Application Publication
(10) Pub. No.: US 2025/0049770 A1
(43) Pub. Date: Feb. 13, 2025

(54) Phosphodiesterase 4 Inhibitor Decreases Thrombosis in Simulated ECMO Model

(71) Applicants: Board of Regents, The University of Texas System, Austin, TX (US); The Government of the United States, as Represented by the Secretary of the Army, Fort Detrick, MD (US)

(72) Inventors: Andrew Duncan Joseph MEYER, San Antonio, TX (US); Andrew P. CAP, San Antonio, TX (US); Ricardo AGUIAR, San Antonio, TX (US)

(21) Appl. No.: 18/680,591

(22) Filed: May 31, 2024

Related U.S. Application Data
(60) Provisional application No. 63/531,934, filed on Aug. 10, 2023.

(51) Int. Cl. A61K 31/44; A61K 31/727; A61M 1/16

(52) U.S. Cl. CPC A61K 31/44; A61K 31/727; A61M 1/1698

(57) ABSTRACT

A method of treating thrombosis in a subject comprising administering an effective amount of a PDE4 inhibitor.

### CROSS-REFERENCE TO RELATED APPLICATIONS

This patent application claims priority to U.S. Provisional Patent Application No. 63/531,934, filed Aug. 10, 2023, the disclosure of which is incorporated in its entirety.

### FIELD OF THE INVENTION

Methods of treating thrombosis and reducing the risk of thrombus formation in subjects, particularly those who are at risk of thrombus formation due to contact with mechanical devices such as extracorporeal life support, left ventricular assist devices, cardiopulmonary bypass, dialysis, or other mechanical devices that can cause thrombin generation.

### BACKGROUND

Lung injury from trauma, sepsis, and chronic disease is a common cause of death in the critically ill. Recent trials document that extracorporeal membrane oxygenation (ECMO) can reduce mortality in patients with severe lung injury (Coombes et al., NEJM, 2018). ECMO can be achieved via a portable artificial heart and lung machine that provide gas exchange outside of the body. However, despite the use of systemic anticoagulants, blood exposure to ECMO components generate excess thrombin, often resulting in thrombus formation, device failure, and life-threatening thromboembolism. These common complications limit the utilization of this lifesaving technology for critically ill patients with organ failure (e.g., trauma, heart disease, or sepsis). Furthermore, current strategies to prevent extracorporeal circuit clot formation have failed to address pump-specific causes of thromboembolism (Chung et al, JACC, 2020). Current therapies to decrease ECMO-induced tissue factor expression are toxic and have narrow therapeutic windows (e.g., coumadin).

### SUMMARY OF THE INVENTION

In an embodiment, a method of treating thrombosis in a subject comprising administering an effective amount of a PDE4 inhibitor.

In a further embodiment of the method, the subject is in contact with a mechanical device that can cause thrombin generation.

In an embodiment of the method, the PDE4 inhibitor used is roflumilast.

In an embodiment of the method, the method is applicable to subjects who have suffered a lung, heart, kidney injury, or a combination thereof.

In an embodiment of the method, the PDE inhibitor can be administered within about 1 to 60 minutes of the lung, heart, kidney injury, or a combination thereof.

In an embodiment of the method, the subject is administered the PDE inhibitor within about 1 to 24 hours of the lung, heart, kidney injury, or a combination thereof.

In an embodiment of the method, the subject is being provided with extracorporeal life support, a left ventricular assist device, cardiopulmonary bypass, dialysis, or a combination thereof.

In an embodiment of the method, the subject is administered the PDE inhibitor within about 1 to 60 minutes of being provided with extracorporeal life support, a left ventricular assist device, cardiopulmonary bypass, dialysis, or a combination thereof.

In an embodiment of the method, the subject is administered the PDE inhibitor within about 1 to 24 hours of being provided with extracorporeal life support, a left ventricular assist device, cardiopulmonary bypass, dialysis, or a combination thereof.

In an embodiment of the method, the PDE4 inhibitor is selected from the group consisting of Apremilast, Roflumilast, Piclamilast, Crisaborole, Cilomilast, Cilostazol, Diazepam, Drotaverine, Gsk256066, Ibudilast, Rolipram, Amrinone, Doxofylline, Luteolin, Mesembrenone, CC-11050, and Zatolmilast.

In an embodiment of the method, the effective amount of the PDE4 inhibitor is between about 0.001 mg/mL and 0.1 mg/mL.

In an embodiment of the method, the effective amount of the PDE4 inhibitor is about 0.001, 0.002, 0.003, 0.004, 0.005, 0.006, 0.007, 0.008, 0.009, 0.01, 0.02, 0.03, 0.04, 0.05, 0.06, 0.07, 0.09, or 0.1 mg/mL.

In an embodiment of the method, the effective amount of the PDE4 inhibitor is about 0.01 mg/mL.

In an embodiment of the method, the effective amount of the PDE4 inhibitor is about 0.01 mg/mL.

In an embodiment of the method, the method further comprises administering an effective amount of heparin.

In an embodiment of the method, the effective amount of heparin is between about 0.1 U/mL and 1.0 U/mL.

In an embodiment of the method, the effective amount of heparin is about 0.1, 0.2, 0.3, 0.4, 0.5, 0.6, 0.7, 0.8, 0.9, or 1.0 U/mL.

In an embodiment of the method, the effective amount of heparin is about 0.5 U/mL.

In an embodiment of the method, the inhibitor is administered orally.

In an embodiment of the method, the inhibitor is administered intravenously.

In an embodiment, a method of providing a mechanical circulation system to a subject comprising: a pump circuit operable to pump a volume of blood of the subject outside the body of the subject; introducing an inhibitor to the volume of blood that inhibits the pro-thrombotic function of leukocytes in the volume of blood; and the pump circuit pumping the volume of blood with the inhibitor.

In an embodiment of the method of providing a mechanical circulation system to a subject, the inhibitor is a PDE4 inhibitor.

In an embodiment of the method of providing a mechanical circulation system to a subject, the PDE4 inhibitor is selected from the group consisting of Apremilast, Roflumilast, Piclamilast, Crisaborole, Cilomilast, Cilostazol, Diazepam, Drotaverine, Gsk256066, Ibudilast, Rolipram, Amrinone, Doxofylline, Luteolin, Mesembrenone, CC-11050, and Zatolmilast.

In an embodiment of the method of providing a mechanical circulation system to a subject, the effective amount of the PDE4 inhibitor is between about 0.001 mg/mL and 0.1 mg/mL.

In an embodiment, the effective amount of the PDE4 inhibitor is about 0.001, 0.002, 0.003, 0.004, 0.005, 0.006, 0.007, 0.008, 0.009, 0.01, 0.02, 0.03, 0.04, 0.05, 0.06, 0.07, 0.09, or 0.1 mg/mL.

In an embodiment of the method of providing a mechanical circulation system to a subject, the effective amount of the PDE4 inhibitor is about 0.01 mg/mL.

In an embodiment of the method of providing a mechanical circulation system to a subject, the effective amount of the PDE4 inhibitor is between about 0.01 mg/mL.

In an embodiment of the method of providing a mechanical circulation system to a subject, the subject has a lung, heart, kidney injury, or a combination thereof.

In an embodiment of the method of providing a mechanical circulation system to a subject, the subject is administered the PDE inhibitor within about 1 to 60 minutes of the lung, heart, kidney injury, or a combination thereof.

In an embodiment of the method of providing a mechanical circulation system to a subject, the subject is administered the PDE inhibitor within about 1 to 24 hours of the lung, heart, kidney injury, or a combination thereof.

In an embodiment of the method of providing a mechanical circulation system to a subject, the subject is being provided with extracorporeal life support, a left ventricular assist device, cardiopulmonary bypass, dialysis, or a combination thereof.

In an embodiment of the method of providing a mechanical circulation system to a subject, the subject is administered the PDE inhibitor within about 1 to 60 minutes of being provided with extracorporeal life support, a left ventricular assist device, cardiopulmonary bypass, dialysis, or a combination thereof.

In an embodiment of the method of providing a mechanical circulation system to a subject, the subject is administered the PDE inhibitor within about 1 to 24 hours of being provided with extracorporeal life support, a left ventricular assist device, cardiopulmonary bypass, dialysis, or a combination thereof.

In an embodiment of the method of providing a mechanical circulation system to a subject, the method further comprises administering an effective amount of heparin.

In an embodiment of the method of providing a mechanical circulation system to a subject, the effective amount of heparin is between about 0.1 U/mL and 1.0 U/mL.

In an embodiment of the method of providing a mechanical circulation system to a subject, the effective amount of heparin is about 0.1, 0.2, 0.3, 0.4, 0.5, 0.6, 0.7, 0.8, 0.9, or 1.0 U/mL.

In an embodiment of the method of providing a mechanical circulation system to a subject, the effective amount of heparin is about 0.5 U/mL.

In an embodiment of the method of providing a mechanical circulation system to a subject, the inhibitor is administered orally.

In an embodiment of the method of providing a mechanical circulation system to a subject, the inhibitor is administered intravenously.

In an embodiment, a mechanical circulation system comprising: a pump circuit operable to pump a volume of blood of a subject outside a body of a subject, wherein introduction of an inhibitor to the volume of blood inhibits pro-thrombotic function of leukocytes in the volume of blood and reduces formation of thrombotic deposits on one or more surfaces of the pump circuit of the mechanical circulation system.

In an embodiment of the mechanical circulation system, the inhibitor is a PDE4 inhibitor.

In an embodiment of the mechanical circulation system, the PDE4 inhibitor is Apremilast, Roflumilast, Piclamilast, Crisaborole, Cilomilast, Cilostazol, Diazepam, Drotaverine, Gsk256066, Ibudilast, Rolipram, Amrinone, Doxofylline, Luteolin, Mesembrenone, CC-11050, Zatolmilast, or a combination thereof.

In an embodiment of the mechanical circulation system, the subject has a lung, heart, kidney injury, or a combination thereof.

In an embodiment of the mechanical circulation system, the system is providing a subject with extracorporeal life support, a left ventricular assist device, cardiopulmonary bypass, dialysis, or a combination thereof.

In an embodiment of the mechanical circulation system, the inhibitor is combined with heparin in the volume of blood.

In an embodiment of the mechanical circulation system, the inhibitor is administered orally.

In an embodiment of the mechanical circulation system, the inhibitor is administered intravenously.

### BRIEF DESCRIPTION OF THE DRAWINGS

FIG. 1 shows a diagram depicting coagulation initiation by an ECMO circuit surface or cell-based Coagulation.

FIG. 2 shows Platelet-Monocyte Induced Tissue Factor: 1. Platelet binds using P-selectin-PSGL to monocyte. 2. Mac-1/ITAM activate Syk. 3. Syk activates PI3K, 4. PI3K phosphorylates Akt, 5. Akt^{P} inhibits GSK3 removing the break on NFkB, 6. NFkB makes TF mRNA, 7. Monocyte express TF, 8. TF+FVIIa+X make thrombin, 9. Thrombin converts fibrinogen to clot. PDE4 inhibitor (roflumilast) prevents cAMP degradation, which blocks Syk activation and platelet activation of monocyte expression of tissue factor.

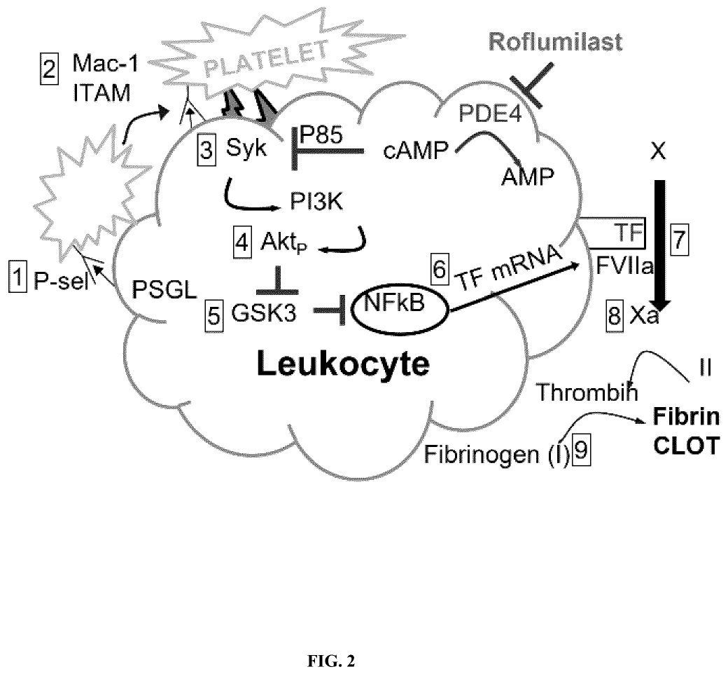

FIG. 3 shows experimental results of P-selection expression under nominal flow (0.5 L/min), low flow (0.3 L/min), low flow with Roflumilast and a static control. Nominal flow and low flow significantly increased P-selection expression over six hours and roflumilast significantly inhibited p-selectin expression (p<0.05).

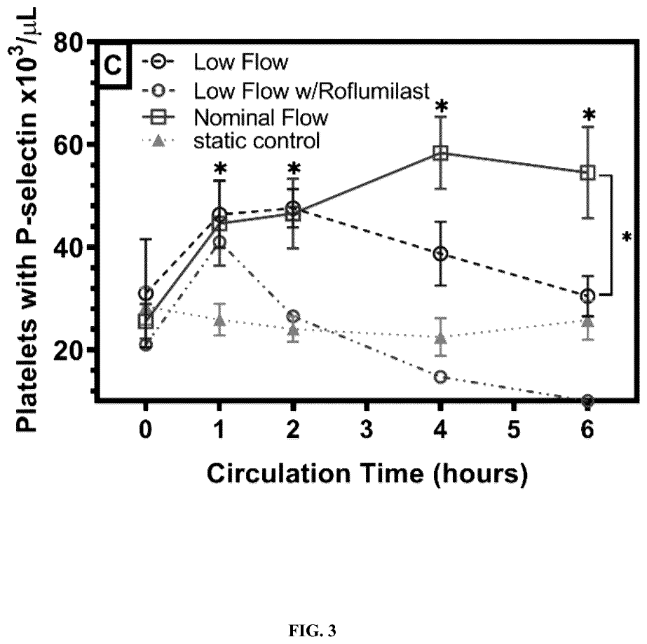

FIG. 4 shows experimental results of monocytes expression of tissue factor under nominal flow (0.5 L/min), low flow (0.3 L/min), low flow with Roflumilast and a static control. Monocytes expression of tissue factor increased more at low flow than circuits treated with roflumilast (p<0.05).

FIG. 5 shows experimental results of whole blood samples treated with heparin, heparin with Roflumilast, leukodepleted blood or static control circulated through a circuit at a nominal flow rate (0.5 L/min) for six hours or until clot formation caused device failure. Samples were collected hourly. Low flow decreased lag time over six hours of circulation less than roflumilast treated circuits.

FIG. 6 shows experimental results of whole blood samples treated with heparin, heparin with Roflumilast, leukodepleted blood or static control circulated through a circuit at a nominal flow rate (0.5 L/min) for six hours or until clot formation caused device failure. Peak thrombin increased significantly more in heparin only compared to leukodepleted or roflumilast and heparin treated circuits (p<0.05).

FIG. 7 shows experimental results of whole blood samples treated with heparin, heparin with Roflumilast, leukodepleted or static control circulated through a circuit at a nominal flow rate (0.5 L/min) for six hours or until clot formation caused device failure. Roflumilast inhibited leukocyte activation as measured by TNF-alpha.

FIG. 8 shows experimental results of whole blood circulated in a simulated Cardiohelp HTS in vitro ECMO system with a double-lumen veno-venous catheter at a low flow rate (1 L/min) for six hours or until clot formation caused device failure. After five hours of circulation, the addition of roflumilast to heparin decreased clot formation as measured by d-dimer from 0.49±0.08 to 0.30±0.06 μg/mL (p<0.04) and increased plasminogen activating inhibitor levels by 1.6-fold more than heparin alone (p<0.03). Preliminary results also document that the addition of roflumilast decreased thromboinflammation generating less monocyte activation, myeloperoxidase, and p-selectin than heparin alone.

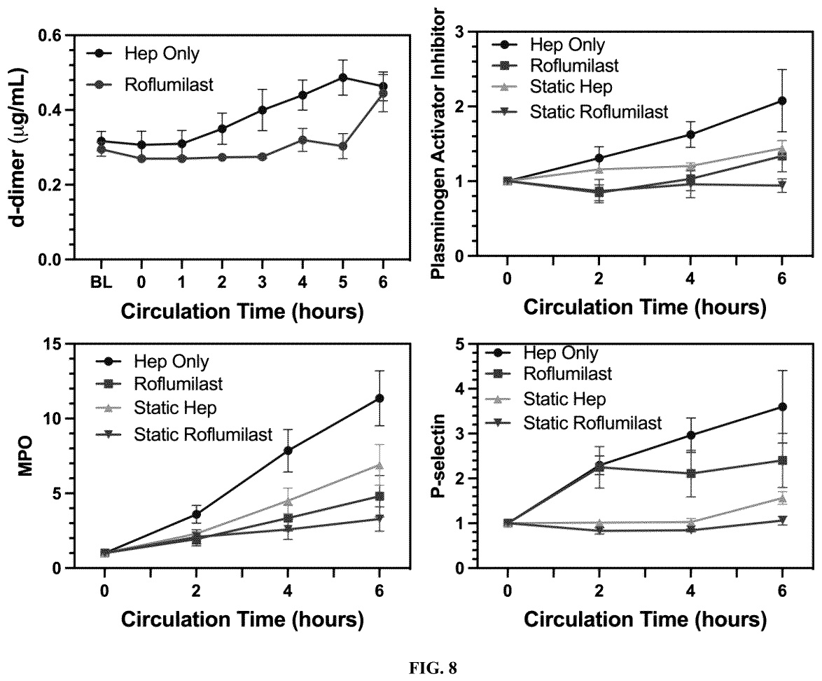

### DETAILED DESCRIPTION

Lung injury from trauma, sepsis, and chronic disease is a common cause of death in the critically ill. Recent trials document that extracorporeal membrane oxygenation (ECMO) can reduce mortality in patients with severe lung injury (Coombes et al., NEJM, 2018). ECMO is a set of devices that include a portable artificial heart and lung machine that can provide gas exchange outside of the body. Despite the use of systemic anticoagulants, blood exposure to ECMO components generate excess thrombin, often resulting in thrombus formation, device failure, and life-threatening thromboembolism. These common complications limit the utilization of this lifesaving technology for critically ill patients with organ failure (e.g., trauma, heart disease, or sepsis). Furthermore, current strategies to prevent extracorporeal circuit clot formation have failed to address pump-specific causes of thromboembolism (Chung et al, JACC, 2020). Current therapies to decrease ECMO-induced tissue factor expression are toxic and have narrow therapeutic windows (e.g., coumadin).

Shear stress induced platelets and platelet extracellular vesicles (EVs) induce tissue factor (TF) expression through P-selectin tethering to P-selectin glycoprotein 1 (PSGL) on monocytes. This monocyte-platelet aggregation induces intracellular cell-signaling to cause a pro-inflammatory and pro-thrombotic state. Christersson et al (Thromb Haemost. 2008), documented that monocyte expression of TF and inflammatory cytokines may be dependent on monocyte-platelets aggregates (MPA). As platelets and EVs bind to monocytes, they mediate intracellular signaling with macrophage-1 (MAC) and immunoreceptor tyrosine-based activation motif (ITAM). These integrins will phosphorylate the non-regulatory tyrosine kinase, spleen tyrosine kinase (Syk), to cause further downstream signaling. Syk then activates phosphoinositide-3 kinase (PI3K) gamma-dependent pathways in the monocyte.

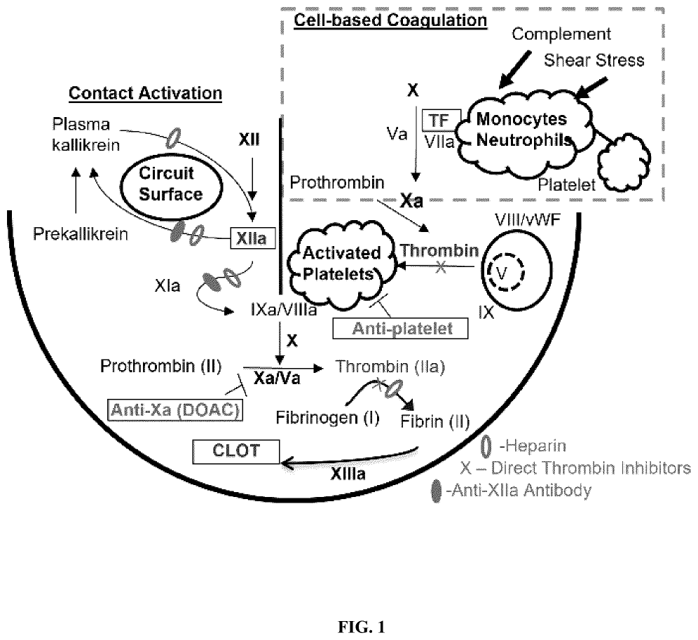

The pathway continues by phosphorylating protein kinase b (Akt), which deactivates glycogen synthase kinase 3 (GSK3), a potent regulator of nuclear factor-kb (NFkB) translation of TF-mRNA and TF expression. FIG. 1 illustrates the pathways that lead to TF messenger RNA (mRNA) upregulation and TF expression in monocytes exposed to activated platelets and EV. Straub and others (Throm. Haemost, 2008) confirmed that PI3K inhibitors are anticoagulants by using a simple extracorporeal circulation loop (e.g., a loop of plastic externally rotated). They documented that a platelet-specific PI3K p100b inhibitor can decrease monocyte-platelet aggregates, platelet loss, and expression of Mac-1 (CD11b/CD18). Di Santo et al (J. Thromb Haemost., 2011) then confirmed and expanded these findings by blocking PI3K with leukocyte-specific wortmannin, preventing NFkB translation of TF-mRNA and TF expression.

However, patients can get resistance to these drugs, and they have increasing reports of severe adverse events. Specifically, idelalisib (a leukocyte-specific PI3K inhibitor), has been linked to transaminitis, opportunistic infections, and pneumonitis.

Theophylline, a non-selective phosphodiesterase (PDE) inhibitor, has successfully been used in asthma and other inflammatory diseases. However, state-of-the-art PDE4 inhibitors can selectively decrease PI3K-mediated expression of pro-thrombotic proteins in monocytes and neutrophils by upregulation of cyclic adenosine monophosphate (cAMP) without significant side effects compared to non-selective PDE inhibitors (theophylline). Increased levels of cAMP within immune cells inhibit the P85 subunit of PI3K, preventing further downstream signaling. Totani et al (J. Thromb Haemost. 2016) determined that the PDE4 inhibitor roflumilast prevents the pro-thrombotic functions of neutrophils and monocytes. Roflumilast, which has FDA approval for severe chronic obstructive pulmonary disease, has other anti-inflammatory effects, including reducing lung and kidney injury. PDE4 inhibitors are generally well tolerated, but psychiatric and gastrointestinal side effects have been reported. New formulations, (e.g., aprelimast), hold promise as safe and effective drugs for thrombo-inflammatory complications.

The present invention addresses the shortcomings of current anticoagulation regimens in mechanical circulation systems, including extracorporeal membrane oxygenation (ECMO), left ventricular assist devices, hemodialysis, and cardiopulmonary bypass. Studies have demonstrated that activated leukocytes promote ECMO thrombosis, and that leukocyte inhibitors, such as phosphodiesterase inhibitor 4s (e.g., roflumilast), can decrease thrombin generation and clot formation. Therefore, the present invention provides for the use of phosphodiesterase inhibitor 4 (PDE4) inhibitors as effective anticoagulants in patients receiving mechanical circulation, improving the anticoagulation regimens for these systems and decreasing complications.

Studies have revealed that activated leukocytes and platelets promote ECMO thrombosis under controlled blood flow conditions. Leukocytes activated by the blood pump generated shear stress produce tissue factor, a potent procoagulant on their surface. Shear-stress activated platelets can also activate leukocytes to express tissue factor. FDA-approved leukocyte inhibitors, developed as an anti-inflammatory for cancer research, were tested under an in vitro ECMO model to determine any anticoagulant effect. Phosphodiesterase inhibitor 4s, is a class of anti-inflammatory medications, that are approved for inflammatory conditions such as chronic obstructive disease. Cell culture experiments document that roflumilast decreases monocyte activation of tissue factor. Our studies document the discovery that PDE4 inhibitors can inhibit leukocyte activation by ECMO systems. In vitro ECMO experiments with roflumilast showed decreased thrombin generation and clot formation. It was discovered that PDE4 inhibitors are effective anticoagulants for patients receiving mechanical circulation including ECMO, left ventricular assist device, dialysis, and cardiopulmonary bypass. This could improve the anticoagulation regiments for these systems, decreasing complications and improving utilization.

Methods of treating thrombosis and reducing the risk of thrombus formation in subjects, particularly those who are at risk of thrombus formation due to contact with mechanical devices such as extracorporeal life support, left ventricular assist devices, cardiopulmonary bypass, dialysis, or other mechanical devices that can cause thrombin generation. Also, methods of reducing thrombotic deposits on surfaces of mechanical circulation systems. The methods comprise administering an inhibitor, either alone or in combination with other drugs such as heparin.

### PDE4 Inhibitors

PDE4 inhibitors include, but are not limited to: Apremilast, Roflumilast, Piclamilast, Crisaborole, Cilomilast, Cilostazol, Diazepam, Drotaverine, Gsk256066, Ibudilast, Rolipram, Amrinone, Doxofylline, Luteolin, Mesembrenone, CC-11050, Zatolmilast, or combinations thereof. The PDE4 inhibitor can be roflumilast. The PDE4 inhibitor may be combined with heparin in the volume of blood.

The effective amount of the PDE4 inhibitor can be between about 0.001 mg/mL and 0.1 mg/mL. The effective amount of the PDE4 inhibitor is about 0.001, 0.002, 0.003, 0.004, 0.005, 0.006, 0.007, 0.008, 0.009, 0.01, 0.02, 0.03, 0.04, 0.05, 0.06, 0.07, 0.09, or 0.1 mg/mL. The effective amount of the PDE4 inhibitor can be about 0.01 mg/mL.

The method can further comprise administering an effective amount of heparin. The effective amount of heparin can be between about 0.1 U/mL and 1.0 U/mL. The effective amount of the heparin can be about 0.1, 0.2, 0.3, 0.4, 0.5, 0.6, 0.7, 0.8, 0.9, or 1.0 U/mL. The effective amount of heparin can be is about 0.5 U/mL.

The PDE4 inhibitor can be formulated in a composition, optionally a pharmaceutical composition. The pharmaceutical composition can comprise a buffer, salt, excipient, solvent, carrier, or a combination thereof. The composition can further comprise an effective amount of heparin.

### Methods of Treatment

A method of treating thrombosis in a subject can comprise administering a PDE4 inhibitor.

A method of providing a mechanical circulation system to a subject, comprising: a pump circuit operable to pump a volume of blood of the subject outside the body of the subject, introducing an inhibitor to the volume of blood that inhibits the pro-thrombotic function of leukocytes in the volume of blood, and the pump circuit pumping the volume of blood with the inhibitor.

A mechanical circulation system comprising: a pump circuit operable to pump a volume of blood of a subject outside a body of a subject, wherein introduction of an inhibitor to the volume of blood inhibits pro-thrombotic function of leukocytes in the volume of blood and reduces formation of thrombotic deposits on one or more surfaces of the pump circuit of the mechanical circulation system.

The subject can be in contact with a mechanical device that can cause thrombin generation. The PDE4 inhibitor can be Apremilast, Roflumilast, Piclamilast, Crisaborole, Cilomilast, Cilostazol, Diazepam, Drotaverine, Gsk256066, Ibudilast, Rolipram, Amrinone, Doxofylline, Luteolin, Mesembrenone, CC-11050, and Zatolmilast. The PDE4 inhibitor may be combined with heparin in the volume of blood.

The subject may be being provided with extracorporeal life support, a left ventricular assist device, cardiopulmonary bypass, dialysis, another mechanical device that can cause thrombin generation, or a combination thereof. The subject may be being provided with extracorporeal life support, a left ventricular assist device, cardiopulmonary bypass, dialysis, another circulatory assist device, or a combination thereof. The subject can be administered the PDE inhibitor within about 1 to 60 minutes of being provided with extracorporeal life support, a left ventricular assist device, cardiopulmonary bypass, dialysis, or a combination thereof. The subject can be administered the PDE inhibitor within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes of being provided with extracorporeal life support, a left ventricular assist device, cardiopulmonary bypass, dialysis, or a combination thereof.

The subject can be administered the PDE inhibitor within about 1 to 24 hours of being provided with extracorporeal life support, a left ventricular assist device, cardiopulmonary bypass, dialysis, or a combination thereof. The subject can be administered the PDE inhibitor within about 1, 2, 3, 4, 5, 6, 7, 8, 9, 10, 11, 12, 13, 14, 15, 16, 17, 18, 19, 20, 21, 22, 23, or 24 hours of being provided with extracorporeal life support, a left ventricular assist device, cardiopulmonary bypass, dialysis, or a combination thereof.

The subject can further be administered heparin within about 1 to 60 minutes of being provided with extracorporeal life support, a left ventricular assist device, cardiopulmonary bypass, dialysis, or a combination thereof. The subject can further be administered heparin within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes of being provided with extracorporeal life support, a left ventricular assist device, cardiopulmonary bypass, dialysis, or a combination thereof.

The subject can further be administered heparin within about 1 to 24 hours of being provided with extracorporeal life support, a left ventricular assist device, cardiopulmonary bypass, dialysis, or a combination thereof. The subject can further be administered heparin within about 1, 2, 3, 4, 5, 6, 7, 8, 9, 10, 11, 12, 13, 14, 15, 16, 17, 18, 19, 20, 21, 22, 23, or 24 hours of being provided with extracorporeal life support, a left ventricular assist device, cardiopulmonary bypass, dialysis, or a combination thereof.

The subject may have a heart, lung, kidney injury, or combination thereof. The subject may have a lung injury. The subject can be administered the PDE inhibitor within about 1 to 60 minutes of suffering the heart, lung, and/or kidney injury, or combination thereof. The subject can be administered the PDE inhibitor within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes of suffering the heart, lung, and/or kidney injury, or combination thereof.

The subject can be administered the PDE inhibitor within about 1 to 24 hours of suffering the heart, lung, and/or kidney injury, or combination thereof. The subject can be administered the PDE inhibitor within about 1, 2, 3, 4, 5, 6, 7, 8, 9, 10, 11, 12, 13, 14, 15, 16, 17, 18, 19, 20, 21, 22, 23, or 24 hours of suffering the heart, lung, and/or kidney injury, or combination thereof.

The subject can further be administered heparin within about 1 to 60 minutes of suffering the heart, lung, and/or kidney injury, or combination thereof. The subject can further be administered heparin within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes of suffering the heart, lung, and/or kidney injury, or combination thereof. The subject can further be administered heparin within about 1 to 24 hours of suffering the heart, lung, and/or kidney injury, or combination thereof. The subject can further be administered heparin within about 1, 2, 3, 4, 5, 6, 7, 8, 9, 10, 11, 12, 13, 14, 15, 16, 17, 18, 19, 20, 21, 22, 23, or 24 hours of suffering the heart, lung, and/or kidney injury, or combination thereof.

The subject is being provided with extracorporeal life support, a left ventricular assist device, cardiopulmonary bypass or dialysis.

The inhibitor can be administered orally. The inhibitor can be administered intravenously.

Further details described herein can be found in the following example, which further defines the scope described herein. All references cited throughout the specification, and the references cited therein, are hereby expressly incorporated by reference in their entirety.

### EXAMPLES

### Example 1

### Trial 1

### Experimental Background

Lung injury from trauma, sepsis, and chronic disease is a common cause of death in the critically ill. Recent trials document that extracorporeal membrane oxygenation (ECMO) can reduce mortality in patients with severe lung injury (Coombes et al., NEJM, 2018). ECMO is a portable artificial heart and lung machine that can provide gas exchange outside of the body. Other mechanical assist devices use mechanical pumps and membrane such as cardiopulmonary bypass, dialysis, or heart assist devices. Despite the use of systemic anticoagulation, blood exposure to mechanical circulatory device components generates excess thrombin, often resulting in thrombus formation, device failure, and life-threatening thromboembolism (Nunez et al, Intensive Care Med, 2022). Our studies document that activated leukocytes promote ECMO thrombosis under controlled flow conditions. Basic laboratory studies document that phosphodiesterase type 4 inhibitor (PDE4) can selectively inhibit the pro-thrombotic function of leukocytes. Roflumilast (a PDE4 inhibitor) may decrease thrombin generation in an ECMO circuit using human blood.

### Experimental Methods

Whole blood was collected from healthy adults under an U.S. Army Institute of Surgical Research approved standard operating procedure. A simulated neonatal roller pump with a KIDS D100 oxygenator circulated human whole blood at a low or nominal flow rate (−0.3 and 0.5 L/min) for six hours or until clot formation caused device failure. Samples were collected hourly from the ECMO model that received heparin alone or heparin plus roflumilast (a PDE4 inhibitor). Expression of pro-thrombotic proteins on leukocytes and extracellular vesicles (EV) were measured with high-resolution flow cytometry. Coagulation factor activity, platelet counts, time to thrombin generation, peak thrombin, and endogenous thrombin potential were quantified.

Whole blood was collected from healthy adult humans into a blood transfer bag containing 0.5 U/mL of heparin with or without roflumilast at 0.01 microgram per mL, under an approved protocol by the U.S. Army Institute of Surgical Research. The blood was then circulated through a roller pump circuit for six hours or until clot formation occurred that prevented forward flow through the circuit. As a control, an aliquot of static blood was removed after priming the circuit and maintained in a similar test environment without circuitry.

Samples were collected hourly from the roller pump circuit, which circulated blood treated with either heparin only or heparin plus roflumilast, at nominal flow rates (0.5 L/min). The expression of pro-thrombotic phospholipids and proteins on platelets, leukocytes, and extracellular vesicles (EV) was measured using high-resolution flow cytometry. Additionally, thromboelastography (TEG) and coagulation indices were used to assess coagulopathy, and a calibrated automated thrombogram was employed to measure differences in whole blood TEG between the roflumilast-treated and heparin-only samples.

### Experimental Results

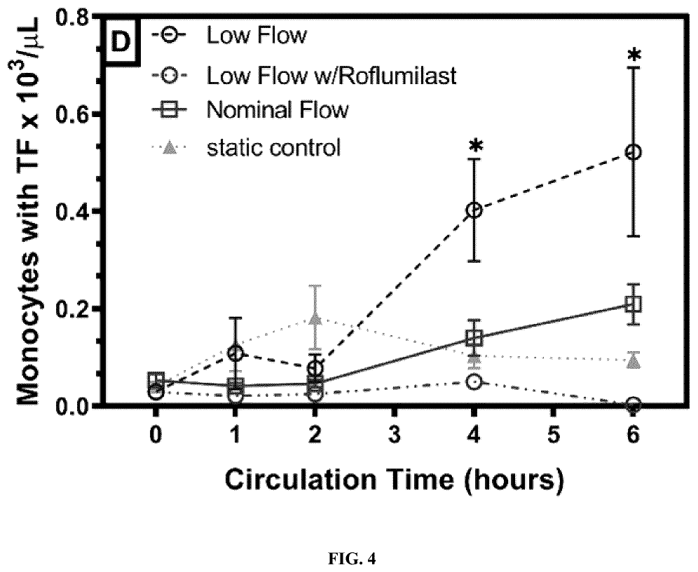

FIG. 4 shows nominal flow (0.5 L/min) and low flow (0.3 L/min) significantly increased P-selection expression over six hours and Roflumilast significantly inhibited p-selectin expression (p<0.05).

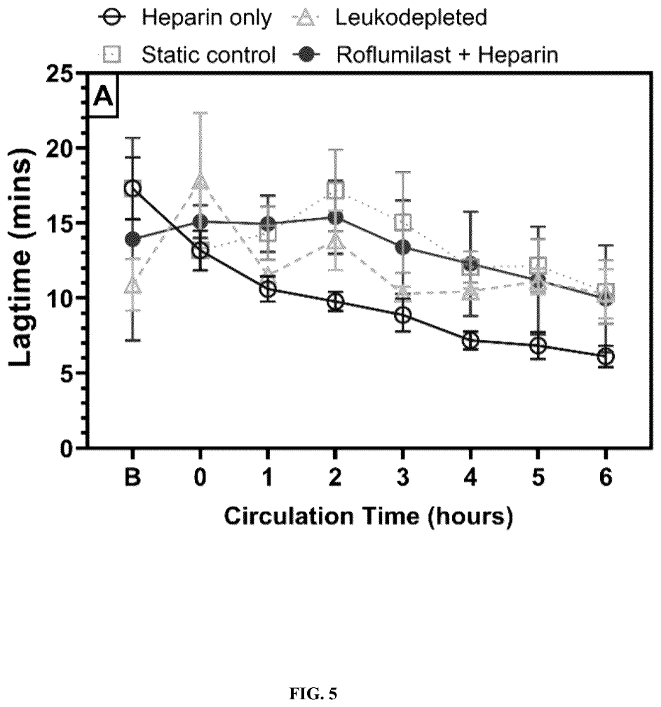

FIG. 5 shows monocytes expression of tissue factor increased more at low flow than circuits treated with Roflumilast (p<0.05).

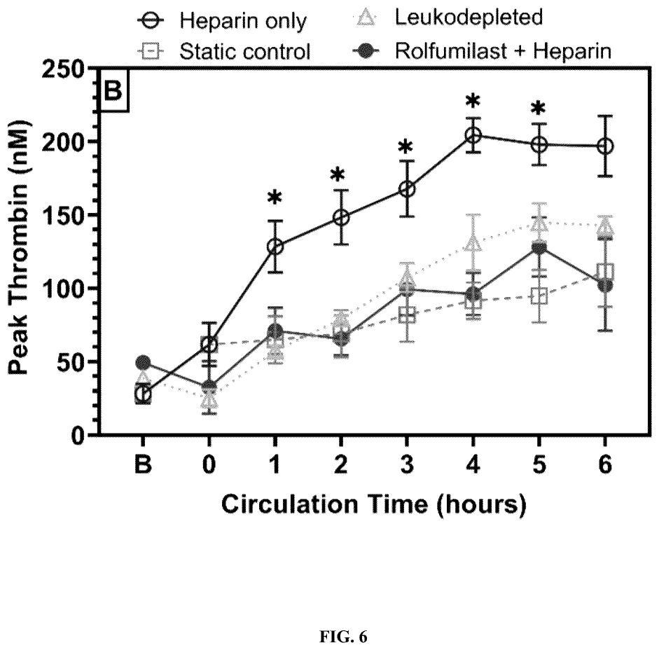

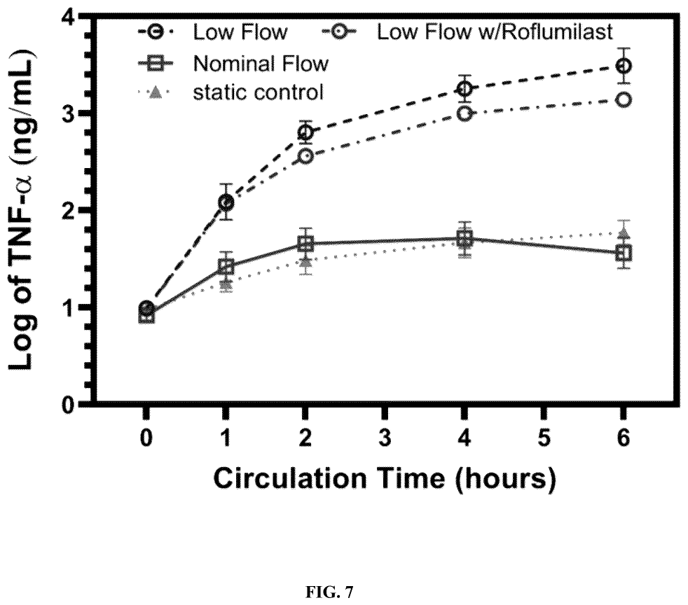

Thrombin Generation: FIG. 6 shows low flow decreased lag time over six hours of circulation less than roflumilast treated circuits. FIG. 7 shows peak thrombin increased significantly more in heparin only compared to leukodepleted or Roflumilast and heparin treated circuits (p<0.05).

The addition of roflumilast to heparin sustained patency in 75% of tested circuits (3/4) after six hours compared to 28% of circuits treated with heparin alone (2/7). Roflumilast decreased extracellular leukocytes expressing tissue factor. Roflumilast plus heparin prolonged time to thrombin generation at 13.9 mins compared to heparin alone at 6.8 mins at the 3-hour time point, (p<0.04). The addition of roflumilast significantly reduced peak thrombin generation compared to heparin alone (69 vs. 207 nm, p<0.02).

Inhibition of leukocyte pro-thrombotic function may be an effective adjunct to decrease thrombotic events during lifesaving mechanical circulatory support for ECMO, dialysis, or heart-assist devices.

### Experimental Conclusion

The present study demonstrates that extracorporeal circulation of human blood activates leukocytes, leading to clot formation. Notably, blocking phosphodiesterase 4 decreased leukocyte activation and thrombin generation in a simulated ECMO circuit with whole blood. These findings suggest that interventions inhibiting immune thrombosis may offer a safe and effective anticoagulant strategy for extracorporeal therapies, ultimately improving the outcomes of patients undergoing these lifesaving treatments.

### Example 2

### Trial 2

### Experimental Methods

Whole blood was collected from healthy adults under an US Army Institution of Surgeryal Review Board approved protocol. A simulated Cardiohelp HTS in vitro ECMO system with a double-lumen veno-venous catheter circulated human whole blood at a low flow rate (1 L/min) for six hours or until clot formation caused device failure. Samples were collected hourly from the ECMO model that received heparin alone or heparin plus roflumilast (a PDE4 inhibitor). Expression of pro-thrombotic proteins on leukocytes and extracellular vesicles (EV) were measured with high-resolution flow cytometry. Coagulation factor activity, platelet counts, time to thrombin generation, peak thrombin, and endogenous thrombin potential were quantified.

### Experimental Results

FIG. 9 shows after five hours of circulation, the addition of roflumilast to heparin decreased clot formation as measured by d-dimer from 0.49±0.08 to 0.30±0.06 μg/mL (p<0.04) and increased plasminogen activating inhibitor levels by 1.6-fold more than heparin alone (p<0.03). Preliminary results also document that the addition of roflumilast decreased thromboinflammation generating less monocyte activation, myeloperoxidase, and p-selectin than heparin alone.

### Experimental Conclusion

Inhibition of thromboinflammation may decrease thrombotic complications in prolonged field care lifesaving ECMO support.

What is claimed is:

1. A method of treating thrombosis in a subject comprising administering an effective amount of a PDE4 inhibitor.

2. The method of claim 1, wherein the subject is in contact with a mechanical device that can cause thrombin generation.

3. The method of claim 1, wherein the PDE4 inhibitor is roflumilast.

4. The method of claim 1, wherein the subject has a lung, heart, kidney injury, or a combination thereof.

5. The method of claim 4, wherein the subject is administered the PDE inhibitor within about 1 to 60 minutes of the lung, heart, kidney injury, or a combination thereof.

6. The method of claim 4, wherein the subject is administered the PDE inhibitor within about 1 to 24 hours of the lung, heart, kidney injury, or a combination thereof.

7. The method of claim 1, wherein the subject is being provided with extracorporeal life support, a left ventricular assist device, cardiopulmonary bypass, dialysis, or a combination thereof.

8. The method of any one of claim 1, wherein the PDE4 inhibitor is selected from the group consisting of Apremilast, Roflumilast, Piclamilast, Crisaborole, Cilomilast, Cilostazol, Diazepam, Drotaverine, Gsk256066, Ibudilast, Rolipram, Amrinone, Doxofylline, Luteolin, Mesembrenone, CC-11050, and Zatolmilast.

9. The method of claim 1, wherein the effective amount of the PDE4 inhibitor is between about 0.001 mg/mL and 0.1 mg/mL.

10. The method of claim 1, wherein the method further comprises administering an effective amount of heparin.

11. A method of providing a mechanical circulation system to a subject comprising:
a pump circuit operable to pump a volume of blood of the subject outside the body of the subject,
introducing an inhibitor to the volume of blood that inhibits the pro-thrombotic function of leukocytes in the volume of blood, and
the pump circuit pumping the volume of blood with the inhibitor.

12. The method of claim 11, wherein the inhibitor is a PDE4 inhibitor.

13. The method of claim 12, wherein the PDE4 inhibitor is selected from the group consisting of Apremilast, Roflumilast, Piclamilast, Crisaborole, Cilomilast, Cilostazol, Diazepam, Drotaverine, Gsk256066, Ibudilast, Rolipram, Amrinone, Doxofylline, Luteolin, Mesembrenone, CC-11050, and Zatolmilast.

14. The method of claim 13, wherein the effective amount of the PDE4 inhibitor is between about 0.001 mg/mL and 0.1 mg/mL.

15. The method of claim 11, wherein the subject has a lung, heart, kidney injury, or a combination thereof.

16. The method of claim 11, wherein the subject is being provided with extracorporeal life support, a left ventricular assist device, cardiopulmonary bypass, dialysis, or a combination thereof.

17. The method of claim 11, wherein the method further comprises administering an effective amount of heparin.

18. A mechanical circulation system comprising:
a pump circuit operable to pump a volume of blood of a subject outside a body of a subject,
wherein introduction of an inhibitor to the volume of blood inhibits pro-thrombotic function of leukocytes in the volume of blood and reduces formation of thrombotic deposits on one or more surfaces of the pump circuit of the mechanical circulation system.

19. The system of claim 18 wherein the inhibitor is a PDE4 inhibitor.

20. The system of claim 18, wherein the PDE4 inhibitor is Apremilast, Roflumilast, Piclamilast, Crisaborole, Cilomilast, Cilostazol, Diazepam, Drotaverine, Gsk256066, Ibudilast, Rolipram, Amrinone, Doxofylline, Luteolin, Mesembrenone, CC-11050, Zatolmilast, or a combination thereof.
